(19) United States
(12) Patent Application Publication
(10) Pub. No.: US 2022/0153820 A1
(43) Pub. Date: May 19, 2022

(54) COMPOSITIONS AND METHODS FOR TREATING AND SUPPRESSING ALLERGIC RESPONSES

(71) Applicant: IgGenix, Inc., South San Francisco, CA (US)

(72) Inventor: Derek Croote, South San Francisco, CA (US)

(21) Appl. No.: 17/529,513

(22) Filed: Nov. 18, 2021

Related U.S. Application Data
(60) Provisional application No. 63/115,503, filed on Nov. 18, 2020.

(51) Int. Cl. C07K 16/16; C07K 16/18; C07K 16/14; C12N 7/00

(52) U.S. Cl. CPC C07K 16/16; C07K 16/18; A61K 48/00; C12N 7/00; C07K 16/14

(57) ABSTRACT

The invention generally relates to therapeutic compositions and methods for treating and suppressing allergic responses.

### TECHNICAL FIELD

The invention generally relates to the fields of medicine, allergies, and immunology, and, more particularly, to therapeutic methods for treating and suppressing allergic responses.

### BACKGROUND

Allergies are characterized by a number of conditions caused by hypersensitivity of the immune system to typically harmless substances in the environment. In general, an allergic reaction occurs when aspects of the immune system overreact to the presence of a substance (an allergen) that, absent the allergy, would not cause a reaction. Food, insect bites, and medications are common causes of severe allergic reactions, with food allergies being the most prevalent. In addition, there are also many significant non-food allergies, including, but not limited to, pollen (e.g., ragweed, trees, and grasses), animals (e.g., animal dander), molds, metals, and latex.

As generally understood, an allergen is a type of antigen that produces an abnormally vigorous immune response in which the immune system fights off a perceived threat that would otherwise be harmless. In technical terms, an allergen is an antigen that is capable of stimulating a type-I hypersensitivity reaction in atopic individuals through Immunoglobulin E (IgE) responses. Most humans mount significant IgE responses only as a defense against parasitic infections. However, some individuals may respond to many common environmental antigens. This hereditary predisposition is called atopy. In atopic individuals, non-parasitic antigens stimulate inappropriate IgE production, leading to type I hypersensitivity.

Some foods such as peanuts (a legume), nuts, seafood and shellfish are the cause of serious allergies in many people. Officially, the United States Food and Drug Administration does recognize eight foods as being common for allergic reactions in a large segment of the sensitive population. These include peanuts, tree nuts, eggs, milk, shellfish, fish, wheat and their derivatives, and soy and their derivatives, as well as sulfites (chemical-based, often found in flavors and colors in foods).

An allergic reaction can be caused by any form of direct contact with the allergen—consuming food or drink one is sensitive to (ingestion), breathing in pollen, perfume or pet dander (inhalation), or brushing a body part against an allergy-causing plant (direct contact). An extremely serious form of an allergic reaction is called anaphylaxis.

Immunoglobulin E (IgE) antibodies mediate the allergic response. They bind to specific receptors on inflammatory immune cells, including mast cells in mucosal tissues lining body surfaces and cavities, as well as basophils in the circulation. These cells mediate allergic responses triggered by specific antigens (allergens) that are recognized by IgE through the release of inflammatory molecules, such as histamine. The inflammatory response is responsible for symptoms, such as sneezing, runny or stuffed nose, itchy eyes, breathing difficulties, and, in extreme cases, anaphylactic shock and even death.

Over the past few decades, the prevalence of food allergies has increased. The most common food allergens include soy products, tree nuts (almond, cashew, walnut, pecan, pistachio, brazil, macadamia, etc.), peanuts, eggs, shellfish, fish, milk, and wheat. Food allergies have a negative impact on quality of life and further results in a significant economic burden. For example, people suffering from allergies are required to be hypervigilant and may avoid situations, including social interactions, that could result in allergic reactions. Furthermore, there appears to be a rise of multi-food allergies, thereby increasing the risk of severe reactions and anaphylaxis.

For many allergies, there is currently no cure and individuals must practice lifelong avoidance. Accordingly, treatments for allergies include the avoidance of known allergens, as well as the use of medications such as steroids and antihistamines. In severe reactions, injectable adrenaline (epinephrine) is recommended as a rescue treatment. One treatment approach for allergies is immunotherapy. Immunotherapy involves the repeated injection or exposure of allergen extracts to desensitize a patient to the allergen. However, immunotherapy is time consuming, usually involving years of treatment, and often fails to achieve its goal of desensitizing the patient to the allergen. Furthermore, immunotherapy carries the risk of potentially severe adverse events, including anaphylaxis.

### SUMMARY

The present invention provides therapeutic methods for treating and suppressing allergic responses. More specifically, the invention encompasses producing high affinity, allergen-specific antibodies designed to alleviate and potentially prevent an allergic response associated with specific allergens. The allergen-specific antibodies may be monoclonal antibodies or may be polyclonal antibodies and may be antigen-binding fragments of the relevant antibody. Whenever the term, “antibody” is used in the disclosure it is intended to mean polyclonal antibodies, monoclonal antibodies, or antigen-binding portions or fragments of any of the foregoing. Preferably, the antibodies are IgG antibodies or antigen-binding fragments thereof having a binding specificity to an associated allergen obtained from an IgE antibody to thereby afford protection (i.e., prevent or suppress allergic response) by stoichiometrically competing with endogenous IgE antibodies to the same allergen. In particular, the allergen-specific antibodies disclosed herein may be configured to block allergen binding to IgE or outcompete endogenous IgE for allergen binding, which in turns prevents or reduces initiation of the allergic cascade. Such antibodies of the present invention are able to provide therapeutic benefits by binding inhibitory receptors on mast cells and/or basophils, for example.

The production of high-affinity, allergen-specific antibodies or fragments may include in vivo production via a vector, such as a viral vector, Cas-mediated introduction in host cells, including bacterial or epithelial cells in the gut, or by other means for the production/expression of the allergen-specific antibodies. Antibodies may be expressed from host cells into which nucleic acids encoding an allergen-specific antibody or antigen-binding fragment thereof are introduced. The expressed allergen-specific, antibody includes at least one heavy chain variable region sequence or light chain variable region sequence derived from an IgE-producing human B cell and/or an IgG producing human B cell, for example. Compositions of the invention may be delivered as protein or as nucleic acid and may be delivered by any suitable means. Moreover, compositions of the invention may be combined with acceptable diluents, carriers, and adjuvants. Thus, in a preferred embodiment, antibodies for use in the invention are class-switching antibodies in which a portion of an IgE antibody is swapped into an IgG antibody as described herein.

An antibody, or antigen-binding fragment thereof, for use in the invention is capable of binding to a known allergen. For example, the specific allergen may include, but is not limited to, a food allergen, a plant allergen, a fungal allergen, an animal allergen, a dust mite allergen, a drug allergen, a cosmetic allergen, or a latex allergen. In some embodiments, antibodies specifically bind to a food allergen, such as a milk allergen, an egg allergen, a nut allergen, a fish allergen, a shellfish allergen, a soy allergen, a legume allergen, a seed allergen, or a wheat allergen. In some embodiments, antibodies specifically bind to a peanut allergen.

In some embodiments, antibodies of the invention are delivered directly in a prolonged release formulation. The antibody itself may be modified to include features that increase serum half-life. Antibodies may be pegylated, conjugated to other proteins (e.g., bovine serum albumen) or provided in a vehicle that causes delayed release of the antibody.

Therapeutic compositions of the invention may comprise an antibody, or antigen-binding portion thereof, formulated for delivery. Delivery may be in oral, intravenous, aerosol or other appropriate formulations. Alternatively, therapeutic compositions of the invention may be delivered in the form of a nucleic acid encoding an appropriate antibody or antigen-binding portion thereof.

In certain aspects, the invention provides methods of preventing or treating an allergic response in a subject. The methods include administering a therapeutically effective amount of a pharmaceutical formulation including a vector that comprises nucleic acids including a nucleic acid sequence encoding an antibody or antigen-binding portion thereof specific to one or more allergens. The antibody may be a monoclonal antibody or an antigen-binding portion thereof. The vector may be a viral vector such as an adeno-associated virus (AAV).

In some embodiments, the includes a heavy chain variable region sequence and a light chain variable region sequence derived from an IgE-producing human B cell associated with the one or more allergens. Preferably the monoclonal antibody comprises a binding specificity to the one or more allergens obtained from the IgE antibody. The monoclonal antibody prevents or suppresses an allergic response by stoichiometrically competing with endogenous IgE antibodies associated with the same one or more allergens.

Methods may include transducing, via the vector, the nucleic acids to one or more host cells. The host cells may be used to produce the allergen-specific antibody. E.g., in some embodiments, the host cells are bacterial cells that express the antibody for use in the method. In certain embodiments, the host cells are epithelial cells. The administering step may include delivering the host cells to the subject.

The allergen may be a food allergen, a plant allergen, a fungal allergen, an animal allergen, a drug allergen, a cosmetic allergen, and a latex allergen. The method may be used to target a food allergen such as a milk allergen, an egg allergen, a nut allergen, a fish allergen, a shellfish allergen, a soy allergen, a legume allergen, a seed allergen, or a wheat allergen. In preferred embodiments, the allergen is one of a peanut allergen, a tree nut allergen, a milk allergen, or a fungal allergen such as an Aspergillus allergen.

Other aspects of the invention provide compositions for treating allergy. Compositions of the invention include a vector comprising nucleic acids that include a sequence encoding an antibody, or at least an antigen-binding portion of the antibody, specific to an allergen and a pharmaceutically-acceptable carrier. The antibody may be a monoclonal antibody or an antigen-binding portion thereof. In the compositions, preferably the antibody includes a heavy chain variable region sequence and a light chain variable region sequence derived from an IgE-producing human B cell associated with allergen. When the composition is delivered to a subject, the antibody prevents or suppresses an allergic response by stoichiometrically competing with endogenous IgE antibodies for the allergen. The allergen may be a food allergen, a plant allergen, a fungal allergen, an animal allergen, a drug allergen, a cosmetic allergen, or a latex allergen. The antibody or antigen-binding portion thereof may bind to an allergen from milk, egg, tree nut, fish, shellfish, soy, legume, seed, wheat, peanut, or fungus. In preferred embodiments, the antibody comprises at least a portion of an IgG antibody (e.g., which does not cross-link Fc receptors on mast cells when the composition is delivered to a subject). The antibody may include one or more Fc mutations that disrupt Fc receptor (FcR) interaction (e.g., the L234A, L235A (LALA) mutations).

In viral embodiments of the compositions, the vector comprises a viral vector such as an adeno-associated virus (AAV), a lentivirus, or an adenovirus.

In DNA embodiments of the compositions, the vector may include non-viral DNA.

In cell expression embodiments, the non-viral DNA may be provided within one or more host cells within the pharmaceutically-acceptable carrier. The non-viral DNA may be synthetic DNA exogenous to the host cell. The one or more host cells may be bacterial cells or epithelial cells. Preferably the one or more host cells transcribe the non-viral DNA and express the antibody, or the antigen-binding portion of the antibody, when the composition is delivered to a subject.

Some of the DNA embodiments use gene-editing system to deliver the nucleic acids encoding the antibody or fragment thereof to a subject. The compositions may include a gene editing system, or nucleic acid encoding the gene editing system, wherein when the composition is delivered to a subject, the gene-editing system inserts the sequence encoding the antibody into a genome of the subject. The gene editing system may include a Cas endonuclease and one or more guide RNAs that specifically hybridize to an insertion locus in the genome. The insertion locus may be a genomic safe harbor such as the adeno-associated virus site 1 (AAVS1) on chromosome 19; the chemokine (C-C motif) receptor 5 (CCR5) gene; and the human ortholog of the mouse Rosa26 locus. In certain embodiments, the gene editing system is included in the pharmaceutically acceptable carrier as ribonucleoproteins (RNPs) comprising Cas endonuclease complexed with guide RNAs that specifically hybridize to an insertion locus in the genome. In some embodiments, the sequence encoding the antibody, or at least the antigen-binding portion of the antibody, are provided within an expression cassette with one or more of a promoter and a transcription factor binding site. For gene-editing embodiments, the sequence encoding the antibody, or at least the antigen-binding portion of the antibody, may include end segments that promote integration of the expression cassette into the genomic safe harbor.

Certain DNA embodiments use plasmids, e.g., the non-viral DNA may include one or more plasmids. The sequence encoding the antibody may be a plasmid DNA-encoded monoclonal antibody (pDNA-mAbs). The pharmaceutically acceptable carrier may be provided within a delivery vessel or reservoir of an electroporation system. The plasmids may include promoters, optionally human cytomegalovirus (CMV) promoters or chicken beta-actin (CAG) promoters. Plasmids may, in the pharmaceutically acceptable carrier, be stable when stored at room temperature. Optionally, the plasmids in the pharmaceutically acceptable carrier are provided in a vessel or reservoir of an injection delivery device, such as one designed for intravenous (IV), subcutaneous (SC), intrathecal (IT), intramuscular (IM), or intradermal (ID) delivery. Compositions of the invention may include an agent that facilitates dispersion of the non-viral DNA through extracellular matrix (ECM), such as a protease, hyaluronidase and/or chondroitinase.

In mRNA embodiments of the compositions, the vector comprises messenger RNA (mRNA). The mRNA may be synthetic in vitro-transcribed (IVT) mRNA. Preferably the mRNA encodes RNA processing structures, including a 5′ cap and a polyadenylation tail.

In certain mRNA embodiments, the mRNA is provided in a lipid nanoparticle (LNP) within the pharmaceutically acceptable carrier. When the composition is delivered to a subject, the LNP promotes delivery of the mRNA into cells of the subject and to ribosomes in the cells. The cells may translate the mRNA into the antibody or portion thereof and release the antibody or portion thereof into systemic circulation. The mRNA may include one or more modified nucleosides (e.g., pseudouridine, 5-methylcytidine, 2′-O-methylcytidine (cm); 2′-O-methylguanosine (gm); 2′-O-methyluridine (um); or 2′-O-methylpseudouridine (fm) to promote stability or inhibit an inflammatory or immune response. The LNP may include a cationic lipid for encapsulating or carrying the mRNA such as 1,2-dioleoyl-sn-glycero-3-phosphoethanolamine (DOPE) or N-[1-(2,3-dioleoyloxy)propyl]-N,N,N-trimethylammonium methyl sulfate (DOTAP).

Aspects of the invention provide a composition for treating allergy. The composition includes at least one antibody or antigen-binding portion thereof specific to an allergen and a pharmaceutically-acceptable carrier. Preferably said antibody or antigen binding portion thereof is a high-affinity antibody with a picomolar disassociation constant (KD). The antibody or antigen binding portion thereof may be a monoclonal antibody. The antibody may include a heavy chain variable region sequence and a light chain variable region sequence derived from an IgE-producing human B cell associated with allergen. The antigen-binding portion of the antibody comprises a binding specificity to the allergen obtained from the IgE antibody. In certain embodiments, the antibody comprises a monoclonal IgG4 antibody. When the composition is delivered to a subject, the antibody prevents or suppresses an allergic response by stoichiometrically competing with endogenous IgE antibodies for the allergen (e.g., a food allergen, a plant allergen, a fungal allergen, an animal allergen, a drug allergen, a cosmetic allergen, and a latex allergen or specifically, an allergen from milk, egg, tree nut, fish, shellfish, soy, legume, seed, wheat, peanut, or fungus). Embodiments of the antibody comprise at least a portion of an IgG antibody. The antibody may have one or more Fc mutations that disrupt Fc receptor (FcR) interaction (e.g., at least the L234A, L235A (LALA) mutations). The antibody or antigen binding portion thereof when delivered to a subject blocks the allergen from binding to IgE or outcompete endogenous IgE for allergen binding, thereby inhibiting anaphylaxis. The antibody or antigen binding portion thereof may be a class-switching antibody in which a portion of an IgE antibody is swapped into an IgG antibody. In some embodiments, the antibody or antigen binding portion thereof specifically binds to a peanut allergen, e.g., at Ara h 2, Ara h 3, or Ara h 6.

In certain embodiments, said antibody or antigen binding portion thereof comprises features that increase serum half-life and/or improve IgE blocking. The antibody or antigen binding portion thereof may be linked to one or a plurality of polyethylene glycol (PEG) units. The antibody or antigen binding portion thereof is conjugated to at least one second protein such as albumin (e.g., bovine serum albumin or human serum albumin).

The antibody or antigen binding portion thereof may be provided in a delayed release vehicle that causes delayed release of the antibody. Suitable delayed release vehicles may include hydrophilic biodegradable protein polymers. The antibody or antigen binding portion thereof and the pharmaceutically acceptable carrier may be provided in a vessel or reservoir of an injection delivery device, e.g., designed for one selected from the list consisting of intravenous (IV), subcutaneous (SC), intrathecal (IT), intramuscular (IM), and intradermal (ID) delivery. The composition may include an agent that facilitates dispersion of the antibody or fragment thereof through extracellular matrix (ECM) such as a protease, hyaluronidase and/or chondroitinase.

In some embodiments, the antibody or antigen binding portion thereof is provided in a lipid nanoparticle (LNP) within the pharmaceutically acceptable carrier. When the composition is delivered to a subject, the LNP promotes delivery of the antibody into tissue of the subject and/or inhibits a subject immune or inflammatory response. The LNP may include a cationic lipid and encapsulating or carrying the antibody or fragment thereof such as 1,2-dioleoyl-sn-glycero-3-phosphoethanolamine (DOPE) or N-[1-(2,3-dioleoyloxy)propyl]-N,N,N-trimethylammonium methyl sulfate (DOTAP).

### DETAILED DESCRIPTION

The present invention is directed to therapeutic methods for treating and suppressing allergic responses, particularly food-related allergies. It should be noted, however, that the methods described herein are useful to prevent and treat all forms of allergies and associated allergens.

The present invention provides a therapy involving producing high affinity, allergen-specific antibodies designed to alleviate and potentially prevent an allergic response associated with specific allergens. The allergen-specific antibodies may include an IgG antibody having a binding specificity to an associated allergen obtained from an IgE antibody to thereby afford protection (i.e., prevent or suppress allergic response) by stoichiometrically competing with endogenous IgE antibodies to the same allergen In particular, the allergen-specific antibodies disclosed herein may be configured to block allergen binding to IgE or outcompete endogenous IgE for allergen binding, which in turns prevents or reduces initiation of the allergic cascade. Such antibodies of the present invention are able to confer therapeutic benefits by binding inhibitory receptors on mast cells and/or basophils, for example.

The production of such high affinity, allergen-specific antibodies may include in vivo production by way of viral vector introduction or Cas-mediated introduction of genetic material into host cells of a patient (e.g., bacterial or epithelial cells in the gut) for the subsequent production/expression of the allergen-specific antibodies. The genetic material includes, for example, nucleic acids comprising a nucleic acid sequence encoding the allergen-specific, antibody. The resulting allergen-specific, antibody includes at least one heavy chain variable region sequence and a light chain variable region sequence derived from an IgE-producing human B cell and/or an IgG producing human B cell, for example.

Methods of the invention provide for the prevention and treatment of an allergic response in a subject. Methods include administering a therapeutically effective amount of a pharmaceutical formulation including a vector that comprises nucleic acids including a nucleic acid sequence encoding an antibody specific to one or more allergens.

Methods of the invention provide for the delivery of therapeutically-effective amounts of substances that compete for binding on mast cells and other receptors to which IgE antibodies may bind. In one embodiment, the therapeutic is delivered via a vector as nucleic acid. The vector may include a viral vector. Many viral vectors or virus-associated vectors are known in the art. Such vectors can be used as carriers of a nucleic acid construct into the cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be integrated into the cellular genome. The constructs may include viral sequences for transfection, if desired. Alternatively, the construct may be incorporated into vectors capable of episomal replication, such as an Eptsein Barr virus (EPV or EBV) vector. The inserted material of the vectors (i.e., components of a CRISPR-Cas system) described herein may be operatively linked to an expression control sequence when the expression control sequence controls and regulates the transcription and translation of that nucleotide sequence. In some examples, transcription of an inserted material is under the control of a promoter sequence (or other transcriptional regulatory sequence) which controls the expression of the recombinant nucleic acid.

In some embodiments, the expression vector is a lentiviral vector. Lentiviral vectors may include a eukaryotic promoter. The promoter can be any inducible promoter, including synthetic promoters. In addition, the lentiviral vectors used herein can further comprise a selectable marker, which can comprise a promoter and a coding sequence for the gRNAs and Cas-related endonucleases. Nucleotide sequences encoding selectable markers are well known in the art.

In some embodiments the viral vector is an adeno-associated virus (AAV) vector. AAV can infect both dividing and non-dividing cells and may incorporate its genome into that of the host cell. One suitable viral vector uses recombinant adeno-associated virus (rAAV).

Methods of making and delivering plasmids and vectors are well known in the art, for example Naso, M., et al., Adeno-Associated Virus (AAV) as a Vector for Gene TherapyAdeno-Associated Virus (AAV) as a Vector for Gene TherapyBioDrugs. 2017; 31(4): 317-334; and Rmamoorth, M., et al., Non-Viral Vectors in Gene Therapy- An Overview, J Clin Diagn Res. 2015 January; 9(1): GE01-GE06, each incorporated by reference herein in their entirety.

Methods of the invention further include transducing, via the vector, the nucleic acids to one or more host cells and producing, via one or more transduced host cells, the allergen-specific antibody. The one or more host cells may include, for example, bacterial or epithelial cells.

It should be noted that the nucleic acids, including a nucleic acid sequence, encoding the allergen-specific antibody, are derived from sequences identified from isolated single B cells from a human subject who is allergic to the specific allergen. Methods of deriving such nucleic acids (for the subsequent production of the allergen-specific antibodies) are described in International PCT Application No. PCT/US2019/032951 (published as WO 2019/222679), the disclosure of which is incorporated by reference herein in its entirety. In particular, such methods include combining single cell RNA sequencing (scRNA-seq) with functional antibody assays to elucidate mechanisms underlying the regulation of IgE and to discover high affinity, cross-reactive allergen-specific antibodies.

As previously described, methods of the present invention provide for the administration of a therapeutically effective amount of a pharmaceutical formulation to a subject for preventing or treating an allergic response in said subject. The formulation generally includes a composition comprising the vector and other components, such as, for example, one or more pharmaceutically acceptable carriers, adjuvants, and/or vehicles appropriate for the particular route of administration for which the composition is to be employed. In some embodiments, the carrier, adjuvant, and/or vehicle is suitable for injection (via a needle of the like) for intravenous, intramuscular, intraperitoneal, transdermal, or subcutaneous administration, as well as a consumable, or spray for related oral and inhalant administrations.

In another embodiment, compositions of the invention are delivered using a Cas endonuclease-mediated delivery system. One can deliver a Cas cassette using appropriate guide RNAs directed at a site of interest in cells for expression of the therapeutic antibody via insertion of a coding sequence in the host cell genome by the Cas enzyme and associate co-factors. Accordingly, administration of the pharmaceutical formulation subsequently results in in vivo production of allergen-specific antibodies via viral vector introduction or Cas-mediated introduction of related genetic material into host cells. As previously noted, the antibody may include an antibody that specifically binds to any known allergen. For example, the specific allergen may include, but is not limited to, a food allergen, a plant allergen, a fungal allergen, an animal allergen, a dust mite allergen, a drug allergen, a cosmetic allergen, or a latex allergen. In some embodiments, the antibody is an antibody that specifically binds to a food allergen, such as a milk allergen, an egg allergen, a nut allergen, a fish allergen, a shellfish allergen, a soy allergen, a legume allergen, a seed allergen, or a wheat allergen. In some embodiments, the antibody specifically binds to a peanut allergen.

Methods of preventing or treating an allergic response in a subject include administering a therapeutically effective amount of a pharmaceutical formulation including a vector that comprises nucleic acids including a nucleic acid sequence encoding an antibody or antigen-binding portion thereof specific to one or more allergens. The antibody may be a monoclonal antibody or an antigen-binding portion thereof. The vector may be a viral vector such as an adeno-associated virus (AAV).

In some embodiments, the includes a heavy chain variable region sequence and a light chain variable region sequence derived from an IgE-producing human B cell associated with the one or more allergens. The monoclonal antibody comprises a binding specificity to the one or more allergens obtained from the IgE antibody. Preferably the monoclonal antibody prevents or suppresses an allergic response by stoichiometrically competing with endogenous IgE antibodies associated with the same one or more allergens, e.g., a food allergen, a plant allergen, a fungal allergen, an animal allergen, a drug allergen, a cosmetic allergen, or a latex allergen. In some embodiments, the one or more allergens is a food allergen selected from the group consisting of a milk allergen, an egg allergen, a nut allergen, a fish allergen, a shellfish allergen, a soy allergen, a legume allergen, a seed allergen, and a wheat allergen.

Methods use compositions for treating allergy that include a vector comprising nucleic acids that include a sequence encoding an antibody, or at least an antigen-binding portion of the antibody, specific to an allergen and a pharmaceutically-acceptable carrier. Methods and compositions of the invention use a vector comprising nucleic acids that include a sequence encoding an antibody, or at least an antigen-binding portion of the antibody, specific to an allergen. Nucleic acids that include a sequence encoding an antibody, or at least an antigen-binding portion of the antibody may be obtained by determining coding sequences for the antibody and synthesizing the nucleic acids or cloning the sequences. For example, in some embodiments, RNA-seq is performed on B cells isolated from the peripheral blood of individuals with an allergy. Use of RNA-Seq allows a cell's gene expression, splice variants, and heavy and light chain antibody sequences to be characterized. Blood may be separated into plasma and cellular fractions; plasma stored and later used for allergen-specific immunoglobin concentration measurements, while the cellular fraction may be enriched for B cells prior to FACS. CD19+ B cells of may be sorted exclusively based on immunoglobulin surface expression, but with an emphasis on IgE and/or IgG4 B cell capture. Isotype identity may be determined from scRNA-seq. B cell capture by such methods avoid stringent requirements on FACS gate purity or the need for complex gating schemes. Single cells may be sorted into wells or other fluid partition, e.g., droplets on a microfluidics platform, and processed using a modified version of the Smart1-seq2 protocol. See Picelli, 2014, Full-length RNA-seq from single cells using Smart-seq2, Nat Protocol 9:171-181, incorporated by reference. Sequencing may be performed on an Illumina NextSeq 500 with 2×150 bp reads to an average depth of 1-2 million reads per cell. Sequencing reads may be aligned and assembled to produce a gene expression count table and/or to reconstruct antibody heavy and light chains, respectively. Using software such as STAR for alignment also facilitates the assessment of splicing within single cells. See Dobin, 2013, STAR: ultrafast universal RNA-seq aligner, Bioinformatics 29:15-21, incorporated by reference. Cells may be stringently filtered to remove those of low quality, putative basophils, and those lacking a single productive heavy and light chain. Isotype identity of each cell may be determined by its productive heavy chain assembly, which avoids misclassification of isotype based on FACS immunoglobulin surface staining. From such sequences, the sequences of antibodies such as IgG4 and/or IgE may be determined. Such sequences may be cloned and reproduced recombinantly. See Dodev, 2014, A tool kit for rapid cloning and expression of recombinant antibodies, Scientific Reports 4:5885, incorporated by reference. Methods of making and purifying antibodies are known in the art and were developed by 1980s as described Harlow and Lane, 1988, Antibodies: A Laboratory Manual, CSHP, Incorporated by reference. Antibodies (e.g., IgG4 and/or IgE) may be isolated or purified using hybridoma technology, wherein isolated B lymphocytes in suspension are fused with myeloma cells from the same species to create monoclonal hybrid cell lines that are virtually immortal while still retaining their antibody-producing abilities. See Harlow and Lane, 1988, Antibodies: A Laboratory Manual, Cold Spring Harbor Laboratory, incorporated by reference. Such hybridomas may be stored frozen and cultured as needed to produce the specific monoclonal antibody. Such monoclonal antibodies may be deployed therapeutically in methods of the invention. Those immunoglobins may exhibit single-epitope specificity and the hybridoma clone cultures provide an unchanging supply over many years. Hybridoma clones may be grown in cell culture for collection of antibodies from the supernatant or grown in the peritoneal cavity of a mouse for collection from ascitic fluid.

Whether by recombinant cloning and expression or by hybridoma technology, immunoglobins may be provided for use in a therapeutic composition.

Once the sequences of one or more antibody, or at least an antigen-binding portion of the antibody, specific to an allergen are known or cloned, known recombinant DNA technology, mRNA synthesis, gene editing, or combinations thereof may be used to produce amounts of nucleic acids that include a sequence encoding an antibody, or at least an antigen-binding portion of the antibody in the desired format (e.g., plasmid, expression cassette, RNA, etc.) as discussed below.

Preferably the antigen-binding portion of the antibody comprises a binding specificity to the allergen obtained from the IgE antibody. When the composition is delivered to a subject, the antibody prevents or suppresses an allergic response by stoichiometrically competing with endogenous IgE antibodies for the allergen. In preferred embodiments, the antibody comprises at least a portion of an IgG antibody (e.g., at least a portion of the antibody is from an IgG4). Optionally the antibody has one or more Fc mutations that disrupt Fc receptor (FcR) interaction such as the L234A, L235A (LALA) mutations.

In some embodiments, the vector is transduced into host cells in vitro. The host cells may include the vector and form a part of the pharmaceutical compositions. E.g., the transduced host cells may produce the allergen-specific antibody. The host cells may be bacterial cells or epithelial cells.

In certain embodiments, the vector comprises non-viral DNA, which may be present as one or more plasmids or expression cassettes, optionally delivered with the use of gene editing systems. For such embodiments, the composition may include a gene editing system, or nucleic acid encoding the gene editing system, wherein when the composition is delivered to a subject, the gene-editing system inserts the sequence encoding the antibody into a genome of the subject.

In some embodiments, the gene editing system include nucleases originally discovered in bacteria. Clustered regularly interspaced short palindromic repeats (CRISPR) were originally found in bacterial genomes under common control with various CRISPR-associated (Cas) proteins. Cas protein 9 (Cas9) has since proven to be an RNA-guided endonuclease useful as a gene editing system when complexed with guide RNA within a ribonucleoprotein (RNP). Cas9 is one Cas endonuclease and other, similar nucleases are known. Natively, the guide RNA included two short single-stranded RNAs, the CRISPR RNA (crRNA) that binds to the target in the target genetic material, and the trans-activating RNA (tracrRNA) that must also be present, although those two RNAs are commonly provided as a single, fused RNA sometimes called a single guide RNA (sgRNA). As used herein, guide RNA (gRNA) refers to either format. Cas9 and gRNA form a ribonucleoprotein (RNP) complex and bind to genomic DNA. The Cas9-gRNA complex scans the genome to identify a protospacer adjacent motif (PAM) and then a genomic DNA sequence adjacent to PAM that matches the gRNA sequence to cleave it. The gRNA-dependent interaction is derived from the base-paring between a gRNA and genomic DNA. In contrast, the gRNA-independent interactions take place between genomic DNA and the amino acid residues of Cas9, including the PAM recognition. Thus, by virtue of the sequence of the gRNA, a Cas RNP cleaves target genetic material in a specific and controllable manner. Sequence-specific cleavage is useful for genome editing by, for example, providing a segment of DNA to be spliced in at the cleavage site by homology-directed repair.

To induce expression of the antibody, a CRISPR-associated (Cas) system may be delivered, along with an expression cassette for the antibody, via the composition. The guide RNAs are designed and synthesized with predetermined targeting sequences and are thus unique reagents having a specific function. In Cas systems, the guide RNAs have sequences unique to a particular target site. The Cas system targets a predetermined site in the genome and provides for the insertion of a coding sequence at that site in the genome. The coding sequence preferably encodes the antibody for fragment thereof. Once the coding sequence is integrated at the predetermined site of the tumor genome (which may be, for example, a genomic safe harbor), the coding sequence, i.e., the antibody, is then expressed.

The gene editing system preferably includes a nuclease (i.e., a protein) such as a Cas endonuclease or a transcription activator like effector nuclease, or a nucleic acid that encodes the nuclease (such as a second expression cassette, plasmid, or other DNA segment for delivery). The nuclease preferably includes one or more nuclear localization signals (NLSs) to promote migration of the nuclease to the nucleus of tumor cells. Even when the nuclease is provided in a nucleic acid, e.g., in mRNA or DNA sense, it still may include the NLSs, in frame with the ORF for the nuclease. NLSs are short polypeptide sequences, e.g., about 10 to 25 amino acids long, and the sequences may be determined by searching literature, e.g., searching a medical library database for recent reports of nuclear localization signals.

The nucleotide sequence of the antibody may be provided in or as an expression cassette. The expression cassette may include a promoter operably linked to the nucleotide sequence of the antibody. The expression of the nucleotide sequence in the expression cassette may be controlled by a constitutive promoter or of an inducible promoter that initiates transcription only when exposed to some particular external stimulus.

In a preferred embodiment, the gene editing system uses Cas endonuclease and guide RNA. For example, the Cas endonuclease may be Cas9 from Streptococcus pyogenes (spCas9). The Cas endonuclease may be complexed with a guide RNA as a ribonucleoprotein (RNP). One of skill in the art may design the gRNA to have a 20-base targeting sequence complementary to an insertion locus.

The target may be a sequence describable as 5′-20 bases-protospacer adjacent motif (PAM)-3′, where the PAM depends on Cas endonuclease (e.g., NGG for Cas9). To insert an exogenous antibody, two Cas RNPs may be used along with a pair of guide RNAs. The RNPs bind to their cognate targets in the genome and introduce double stranded breaks. The exogenous nucleic acid sequence to be inserted may have ends that are homologous to sequences flanking the genome to induce the cell's endogenous homology-directed repair response, to repair the genome by inserting the exogenous DNA segment. See How, 2019, Inserting DNA with CRISPR, Science 365(6448):25 and Strecker, 2019, RNA-guided DNA insertion with CRISPR-associated transposases, Science 365(6448):48, both incorporated herein by reference. A Cas endonuclease may be Cas9 (e.g., spCas9), Cpf1 (aka Cas12a), C2c2, Cas13, Cas13a, Cas13b, e.g., PsmCas13b, LbaCas13a, LwaCas13a, AsCas12a, PfAgo, NgAgo, CasX, CasY, others, modified variants thereof, and similar proteins or macromolecular complexes.

The gene editing system may be used to insert the non-viral DNA into a genome of the subject at an insertion locus such as a genomic safe harbor. The gene editing system may be in the pharmaceutically acceptable carrier as ribonucleoproteins (RNPs) comprising Cas endonuclease complexed with guide RNAs that specifically hybridize to an insertion locus in the genome. Here, the sequence encoding the antibody, or at least the antigen-binding portion of the antibody, may include an expression cassette with one or more of a promoter and a transcription factor binding site. The sequence encoding the antibody, or at least the antigen-binding portion of the antibody, may further end segments that promote integration of the expression cassette into the genomic safe harbor.

In some embodiments, the non-viral DNA comprises one or more plasmids. The sequence encoding the antibody may comprise a plasmid DNA-encoded monoclonal antibody (pDNA-mAbs). Plasmids are well suited to intravenous (IV), subcutaneous (SC), intrathecal (IT), intramuscular (IM), or intradermal (ID) delivery and well suited to us with electroporation. Especially for plasmid delivery, the composition may include an agent that facilitates dispersion of the non-viral DNA through extracellular matrix (ECM) such as a protease, hyaluronidase and/or chondroitinase.

In some embodiments of the compositions, the vector comprises messenger RNA (mRNA).

Compositions may include RNA. The RNA may be synthesized by solid-phase synthesis. Solid-phase synthesis may be carried out on a solid support that may be held between filters, in columns that enable all reagents and solvents to pass through freely. With solid-phase synthesis, a large excesses of solution-phase reagents can be used to drive reactions quickly to completion. Preferred embodiments include the phosphoramidite method using solid-phase technology and automation. Phosphoramidite RNA synthesis is used field using synthetic RNA. See McBride, 1983, An investigation of several deoxynucleoside phosphoramidites useful for synthesizing deoxyoligonucleotides. Tetrahedron Lett 24:245-248; and Kosuri, 2014, Large-scale de novo DNA synthesis: technologies and applications Nat Meth 11:499-507, both incorporated by reference.

In some embodiments, mRNA is made by in vitro transcription. In vitro transcription uses a purified linear DNA template containing a promoter, ribonucleotide triphosphates, a buffer system that includes DTT and magnesium ions, and an appropriate phage RNA polymerase. The DNA template preferably includes a double-stranded promoter for binding of the phage polymerase. The template may include plasmid constructs engineered by cloning, cDNA templates generated by first- and second-strand synthesis from an RNA precursor, or linear templates generated by PCR or by annealing chemically synthesized oligonucleotides. The template may be an (e.g., linearized) plasmid. Many plasmids include phage polymerase promoters. Any suitable promoter may be used, e.g., the promoter for any of three common polymerases, SP6, T7 or T3, may be used.

DNA is then transcribed by a T7, T3 or SP6 RNA phage polymerase in the presence of ribonucleoside triphosphates (rNTPs). The polymerase traverses the template strand and uses base pairing with the DNA to synthesize a complementary RNA strand (using uracil in the place of thymine). The RNA polymerase travels from the 3′→5′ end of the DNA template strand, to produce an RNA molecule in the 5′→3′ direction. See Jani, 2012, In vitro Transcription and Capping of Gaussia Luciferase mRNA Followed by HeLa Cell Transfection, J Vis Exp 61:3702, incorporated by reference.

The mRNA may be provided in a lipid nanoparticle (LNP) within the pharmaceutically acceptable carrier. The mRNA may be packaged in (a plurality of) nanoparticles comprising a cationic lipid. Methods for preparation may include direct mixing between cationic liposomes and mRNA in solution, or rehydration of a thin-layer lipid membrane with mRNA in solution. The dispersion of cationic lipid/mRNA complexes in the aqueous solution often results in heterogeneous complexes, sometimes still referred to as cationic liposomes, aka lipoplexes. Lipoplexes can encapsulate nucleic acid cargos up to 90% of the input dose. See Wang, 2015,

Delivery of oligonucleotides with lipid nanoparticles, Adv Drug Deliv Rev 87:68-80, incorporated by reference.

In some embodiments, mRNA or modified mRNA (e.g., prepared with a T7 polymerase-based IVT kit with a yield of ˜60 μg/reaction) interact electrostatically with a preformed DOTAP (1,2-dioleoyl-3-trimethylammonium-propane)/cholesterol (1:1 molar ratio) liposome. Electrostatic interaction between the cationic lipid head group and the backbone of nucleic acids drives encapsulation of mRNA in cationic liposomes. This yields a self-assembly, liposome-based, core membrane nanoparticle formulation. The electrostatic interaction promotes the self-assembly by inducing lipid bilayers to collapse on the core structure, resulting in spherical, solid, liposomal nanoparticles with a core/membrane structure. See Wang, 2013, Systematic delivery of modified mRNA encoding herpes simplex virus 1 thymidine kinase for targeted cancer gene therapy, Mol Ther 21(2):358-367, incorporated by reference.] Thus, in some embodiments, the nanoparticle further comprises N-[1-(2,3-dioleoyloxy)propyl]-N,N,N-trimethylammonium methyl sulfate (DOTAP). The nanoparticles may include 1,2-dioleoyl-sn-glycero-3-phosphoethanolamine (DOPE).

Methods for preparation may include direct mixing between cationic liposomes and RNA in solution, or rehydration of a thin-layer lipid membrane with RNA in solution. The dispersion of cationic lipid/RNA complexes in the aqueous solution may result in heterogeneous complexes, sometimes still referred to as cationic liposomes, aka lipoplexes. Lipoplexes can encapsulate nucleic acid cargos up to 90% of the input dose. See Wang, 2015, Delivery of oligonucleotides with lipid nanoparticles, Adv Drug Deliv Rev 87:68-80, incorporated by reference.

In certain embodiments, HPLC-purified 1-methylpseudouridine-containing mRNA may be encapsulated in LNPs using a self-assembly process. LNPs are prepared using ionizable lipid L319, distearoylphosphatidylcholine (DSPC), cholesterol and PEG-DMG at a molar ratio of 55:10:32.5:2.5 (L319:DSPC:cholesterol:PEG-DMG). The mRNA is introduced at a lipid nitrogen to siRNA phosphate ratio of 3, corresponding to a total lipid to mRNA weight ratio of ˜10:1. A spontaneous vesicle formation process is used to prepare the LNPs. Methods may be used as described in Maier, 2013, Biodegradable lipids enabling rapidly eliminating lipid nanoparticles for systemic delivery of RNAi therapeutics, Mol Ther 21(8):1570-1578; WO 2016/089433 A1, WO 2015/006747 A2, WO 2014/093924 A1, or WO 2013/052523 A1, all incorporated by reference.

A lipid nanoparticle (LNP) may include a gene editing system. LNPs may be about 100-200 nm in size and may optionally include a surface coating of a neutral polymer such as PEG to minimize protein binding and unwanted uptake. The nanoparticles are optionally carried by the pharmaceutically acceptable carrier, such as water, an aqueous solution, suspension, or a gel. For example, LNPs may be included in a formulation that may include chemical enhancers, such as fatty acids, surfactants, esters, alcohols, polyalcohols, pyrrolidones, amines, amides, sulfoxides, terpenes, alkanes and phospholipids. LNPs may be suspended in a buffer. The buffer may include a penetration enhancing agent such as sodium lauryl sulfate (SLS). SLS is an anionic surfactant that enhances penetration into the skin by increasing the fluidity of epidermal lipids. Lipid nanoparticles may be delivered via a gel, such as a polyoxyethylene-polyoxypropylene block copolymer gel (optionally with SLS). LNPs may be freeze-dried (e.g., using dextrose (5% w/v) as a lyoprotectant), held in an aqueous suspension or in an emulsification, e.g., with lecithin, or encapsulated in LNPs using a self-assembly process. LNPs may be prepared using ionizable lipid L319, distearoylphosphatidylcholine (DSPC), cholesterol and PEG-DMG at a molar ratio of 55:10:32.5:2.5 (L319:DSPC:cholesterol:PEG-DMG). The payload may be introduced at a total lipid to payload weight ratio of ˜10:1. A spontaneous vesicle formation process is used to prepare the LNPs. Payload is diluted to ˜1 mg/ml in 10 mmol/l citrate buffer, pH 4. The lipids are solubilized and mixed in the appropriate ratios in ethanol. Payload-LNP formulations may be stored at −80° C. See Maier, 2013, Biodegradable lipids enabling rapidly eliminating lipid nanoparticles for systemic delivery of RNAi therapeutics, Mol Ther 21(8):1570-1578, incorporated by reference. See, WO 2016/089433 A1, incorporated by reference herein.

Compositions of the disclosure may include a plurality of lipid nanoparticles having the nucleic acids encoding the antibody embedded therein. In one embodiment, a plurality of lipid nanoparticles comprises at least a solid lipid nanoparticle comprising a segment of DNA or mRNA that encodes the antibody for fragment thereof; optionally a second solid lipid nanoparticle that includes at least one Cas endonuclease complexed with a gRNA that targets the CRISPR/Cas system to a locus in the genome.

According to compositions and methods of the disclosure, monoclonal antibodies may be used based on ones discovered from human allergic donors. Compositions of the invention use high affinity (e.g., picomolar KD) monoclonal Abs (mAbs). Embodiments of the method use mAbs optionally multiple different mAbs in combination to inhibit allergen-mediated cellular degranulation in vivo. The compositions may be administered to block allergic patient sera IgE from binding to allergen with sub-nM IC50. Preferably the method inhibits activation of IgE sensitized basophil and/or mast cell exposed to allergen by >70% with sub-nM IC50. Certain embodiments include subcutaneous administration. Benefits of the disclosed antibodies may include predictable, minimal toxicities with no human tissue cross-reactivity. Antibodies can be produced in animals, i.e., by immunization of an animal with an allergen. Once the sequence of the allergen is known, it can be cloned, e.g., into yeast or bacteria, and grown up in bulk to form a protein product that primarily includes the allergen for use in animal immunization to raise blocking antibodies. The protein product can be harvested from the growth vector and inoculated into animals (e.g., mice) to cause them to grow antibodies against the allergen. Those antibodies may be harvested and optionally sequenced and/or cloned via hybridoma technology for further expansion, e.g., followed by isolation for use in a therapeutic composition.

Throughout the present description it is understood that methods of the inventions may be used to respond to, study, or treat allergies to any allergens such as from nuts, fish, milk, etc., as well as venoms, pollens, dander, latex, fungi, medicines (including antibiotics) and in particular peanut, milk, shellfish, tree nuts, egg, fin fish, wheat, soy, and sesame.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method of preventing or treating an allergic response in a subject, the method comprising:
administering a therapeutically effective amount of a pharmaceutical formulation including a vector that comprises nucleic acids including a nucleic acid sequence encoding an antibody or antigen-binding portion thereof specific to one or more allergens.

2. The method of claim 1, wherein said antibody is a monoclonal antibody or an antigen-binding portion thereof.

3. The method of claim 1, wherein the vector comprises a viral vector.

4. The method of claim 3, wherein the viral vector is an adeno-associated virus (AAV).

5. The method of claim 2, wherein the monoclonal antibody includes a heavy chain variable region sequence, and a light chain variable region sequence derived from an IgE-producing human B cell associated with the one or more allergens.

6. The method of claim 5, wherein the monoclonal antibody comprises a binding specificity to the one or more allergens obtained from the IgE antibody.

7. The method of claim 6, wherein the monoclonal antibody prevents or suppresses an allergic response by stoichiometrically competing with endogenous IgE antibodies associated with the same one or more allergens.

8. The method of claim 1, further comprising transducing, via the vector, the nucleic acids to one or more host cells.

9. The method of claim 8, further comprising producing, via one or more transduced host cells, the allergen-specific antibody.

10. The method of claim 1, wherein the one or more host cells are bacterial cells.

11. The method of claim 1, wherein the one or more host cells are epithelial cells.

12. The method of claim 1, wherein the one or more allergens comprises at least one of a food allergen, a plant allergen, a fungal allergen, an animal allergen, a drug allergen, a cosmetic allergen, and a latex allergen.

13. The method of claim 12, wherein the one or more allergens is a food allergen selected from the group consisting of a milk allergen, an egg allergen, a nut allergen, a fish allergen, a shellfish allergen, a soy allergen, a legume allergen, a seed allergen, and a wheat allergen.

14. The method of claim 13, wherein the food allergen is a peanut allergen.

15. The method of claim 13, wherein the food allergen is a tree nut allergen.

16. The method of claim 12, wherein the food allergen is a milk allergen.

17. The method of claim 12, wherein the allergen is a fungal allergen.

18. The method of claim 17, wherein the fungal allergen is an Aspergillus allergen.

19-93. (canceled)
